(19) United States
(12) Patent Application Publication
(10) Pub. No.: US 2016/0137968 A1
(43) Pub. Date: May 19, 2016

(54) TISSUE CULTURE TESTING SYSTEMS AND METHODS OF USE

(71) Applicant: Barry University, Miami Shores, FL (US)

(72) Inventors: Gerhild Packert, Pembroke Pines, FL (US); Daniel Packert, Pembroke Pines, FL (US); Stephen Dunham, Tamarac, FL (US); Christopher C. Miller, North Vancouver (CA)

(21) Appl. No.: 14/939,816

(22) Filed: Nov. 12, 2015

Related U.S. Application Data
(60) Provisional application No. 62/079,458, filed on Nov. 13, 2014.

(51) Int. Cl. C12M 1/34; C12M 1/12

(52) U.S. Cl. CPC C12M 41/46; C12M 41/34; C12M 25/02

(57) ABSTRACT

Embodiments of the present disclosure provide materials and methods relating to cell and tissue culture testing systems. Certain embodiments of the present disclosure relate to in vitro testing systems that are useful for performing experiments to investigate the potential for various factors to reduce bioburden, reduce the manifestations of infection, and to promote wound healing in cultured cells and tissues. In some embodiments, the present disclosure provides means for investigating biological mechanisms underlying wound healing, including the ability of pressurized gas to reduce the manifestations of infection by reducing bioburden.

### RELATED APPLICATIONS

The instant application claims the benefit of U.S. Provisional Patent Application Ser. No. 62/079,458, filed Nov. 13, 2014. This application is incorporated herein by reference in its entirety for all purposes.

### STATEMENT REGARDING FEDERALLY FUNDED RESEARCH

Embodiments disclosed herein have been supported in part by Defense Advanced Research Projects Agency (DARPA), DARPA Grant No. HR 0011-11-1-0006. The government has certain rights to this invention.

### FIELD

Embodiments of the present disclosure provide materials and methods relating to cell and tissue culture testing systems. Certain embodiments of the present disclosure relate to in vitro testing systems useful for performing experiments to investigate and/or validate the potential for various factors and/or conditions to reduce bioburden, reduce infections, reduce manifestations of infection, and to promote wound healing in cultured cells and tissues. In certain embodiments, systems and methods disclosed herein can provide validation for potential use of the tested factors and/or conditions to be used in an in vivo setting.

### BACKGROUND

Cell culture and tissue culture systems and methods have evolved to provide physiologically accurate modelling of various biological functions. In vitro studies have led to the isolation, growth and identification of microorganisms, the discovery of new cell types derived from multicellular organisms (e.g., cell culture or tissue culture), the identification of subcellular components (e.g., mitochondria or ribosomes), the isolation of cellular and subcellular extracts (e.g., wheat germ or reticulocyte extracts), the purification of therapeutic small molecules and biologic drugs, and the commercial production of antibiotics and other pharmaceutical products. Generally, studies that are conducted using components of an organism that have been isolated from their physiological or biological context permit a more detailed or more convenient analysis than can be done with whole organisms.

To enable in vitro study of dermal phenomena (e.g., paracrine signaling among keratinocytes), full-thickness skin models have been developed. These models typically consist of normal, human-derived epidermal keratinocytes (NHEK) and normal, human-derived dermal fibroblasts (NHFB), which have been cultured to form a multilayered, highly differentiated model of the human dermis and epidermis. NHEK and NHFB can be cultured on specially prepared cell culture inserts using serum free medium, and can attain similar levels of differentiation seen in vivo. Ultrastructurally, the full-thickness in vitro skin models closely parallel human skin, thus providing a useful means to assess the various therapeutic parameters affecting the physiological and biochemical processes taking place in the skin, such as wound healing.

In vitro systems offer the potential to decipher the biological mechanisms underlying wound healing, including the role that Nitric Oxide (NO) plays in this process. NO produced by both iNOS and eNOS plays many important roles in wound healing, from the inflammatory phase through to scar remodeling. In particular, NO exerts cytostatic, chemotactic, and vasodilatory effects during early wound repair, regulates proliferation and differentiation of several cell types, modulates collagen deposition and angiogenesis, and affects wound contraction. However, the timing, concentration, pressurization, and site of NO administration are all poorly understood critical factors affecting the ability of NO to reduce infection and promote wound healing.

### SUMMARY

Embodiments of the present disclosure provide materials and methods relating to cell and tissue culture testing systems. Certain embodiments of the present disclosure relate to in vitro testing systems that are useful for performing experiments to investigate the potential for various factors to reduce bioburden, reduce the manifestations of infection, and to promote wound healing in cultured cells and tissues.

Embodiments of the present disclosure provide tissue culture testing systems. In some embodiments, the tissue culture testing systems include a gaseous nitric oxide (gNO) delivery device, a tissue culture testing apparatus, the tissue culture testing apparatus comprising at least one top chamber and at least one bottom chamber, and a membrane separating the at least one top chamber from the at least one bottom chamber. According to these embodiments, the gNO delivery device is functionally coupled to the tissue culture testing apparatus and delivers pressurized gNO to a tissue sample.

The system according to paragraph [0008], further comprising at least one gas flow regulator and at least one gas pressure regulator.

The system according to either paragraph [0008] or [0009], further comprising a source of gNO functionally coupled to the gNO delivery device.

The system according to any one of paragraphs [0008]-[0010], wherein the gNO delivery device further comprises one or more nitric oxide sensors.

The system according to any one of paragraphs [0008]-[0011], wherein the gNO delivery device further comprises one or more oxygen sensors.

The system according to any one of paragraphs [0008]-[0012], wherein the top chamber of the tissue culture testing apparatus comprises at least one gas inlet and at least one gas outlet.

The system according to any one of paragraphs [0008]-[0013], wherein the bottom chamber of the tissue culture testing system comprises two or more subchambers.

The system according to any one of paragraphs [0008]-[0014], wherein the membrane separating the top chamber and the bottom chamber of the tissue culture testing apparatus is coupled to a tissue interface insert that engages the bottom chamber of the tissue culture testing apparatus.

The system according to any one of paragraphs [0008]-[0015], wherein the membrane comprises the tissue sample, and wherein the tissue sample is a full-thickness skin tissue sample.

The system according to any one of paragraphs [0008]-[0016], wherein the tissue sample is a full-thickness skin tissue sample, and wherein the full-thickness skin tissue sample is infected with one or more pathogens.

The system according to any one of paragraphs [0008]-[0017], wherein the tissue sample is a full-thickness skin tissue sample, and wherein the full-thickness skin tissue sample is infected with one or more bacterial pathogens.

The system according to any one of paragraphs [0008]-[0018], wherein the pressure of the gNO delivered to the tissue sample is from about 0.15 ATM to about 1.0 ATM.

The system according to any one of paragraphs [0008]-[0019], wherein the gNO is delivered to the tissue sample at a flow rate from about 0.1 liters/minute to about 1.0 liters/minute.

The system according to any one of paragraphs [0008]-[0020], wherein the concentration of the gNO delivered to the subject is about 1.0%.

The system according to any one of paragraphs [0008]-[0021], wherein the gNO is delivered to the tissue sample for about 30 minutes to about 120 minutes.

The system according to any one of paragraphs [0008]-[0022], wherein delivering pressurized gNO to the tissue sample reduces bioburden or reduces one or more manifestations of infection in the tissue sample.

Embodiments of the present disclosure also include methods for determining bioburden reduction in a tissue sample. In some embodiments, the method includes placing at least one tissue sample on a membrane of a tissue culture testing apparatus, the membrane separating at least one top chamber from at least one bottom chamber of the tissue culture testing apparatus, infecting the at least one tissue sample with one or more pathogens, delivering pressurized gaseous nitric oxide (gNO) to the at least one infected tissue sample using a gNO delivery device according to a predetermined experimental protocol, the gNO delivery device functionally coupled to the tissue culture testing apparatus, and determining bioburden reduction in the at least one infected tissue sample.

The method according to paragraph [0024], wherein the tissue sample is a full-thickness skin tissue sample.

The method according to either paragraph [0024] or [0025], the one or more pathogens is one or more bacterial pathogens.

The method according to any of paragraphs [0024]-[0026], wherein the at least one tissue sample comprises at least one treated tissue sample and at least one untreated control tissue sample.

The method according to any of paragraphs [0024]-[0027], delivering gNO according to a predetermined experimental protocol comprises delivering gNO at certain concentrations, flow rates, and pressures.

The method according to any of paragraphs [0024]-[0028], wherein the at least one tissue sample comprises at least one treated tissue sample and at least one untreated control tissue sample, and wherein the method further comprises delivering gNO to the at least one treated tissue sample and delivering air to the at least one untreated control tissue sample.

The method according to any of paragraphs [0024]-[0029], wherein the at least one tissue sample comprises at least one treated tissue sample and at least one untreated control tissue sample, and wherein the method further comprises delivering gNO to both the at least one treated tissue sample and the at least one untreated control tissue sample.

The method according to any of paragraphs [0024]-[0030], wherein delivering gNO to both the at least one treated tissue sample and the at least one untreated control tissue sample according to a predetermined experimental protocol comprises delivering gNO at certain concentrations, flow rates, and pressures.

The method according to any of paragraphs [0024]-[0031], wherein the at least one tissue sample comprises at least one treated tissue sample and at least one untreated control tissue sample, and wherein determining bioburden reduction in the at least one treated tissue sample comprises comparing the at least one treated tissue sample to the at least one untreated control tissue sample prior to, and upon completion of, the predetermined experimental protocol.

The method according to any of paragraphs [0024]-[0032], wherein the at least one tissue sample comprises at least one treated tissue sample and at least one untreated control tissue sample, and wherein determining bioburden reduction in the at least one treated tissue sample comprises comparing total CFUs from the at least one treated tissue sample to total CFUs from the at least one untreated control tissue sample prior to, and upon completion of, the predetermined experimental protocol.

The terms “determine,” “calculate,” and “compute,” and variations thereof, as used herein, are used interchangeably and include any type of methodology, process, mathematical operation or technique.

It is to be noted that the term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “means” as used herein shall be given its broadest possible interpretation in accordance with 35 U.S.C. §112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a representation of a tissue culture testing system that includes a gaseous Nitric Oxide (gNO) delivery device and a plurality of Franz cells attached in series, according to an embodiment of the present disclosure.

FIG. 2 is a representation of an individual Franz cell, according to an embodiment of the present disclosure.

FIGS. 3A-3C are representations of various Franz cell chambers, including a Franz cell chamber for testing single tissue samples (FIG. 3A), a Franz cell chamber for testing three tissue samples (FIG. 3B), and a Franz cell chamber that includes a tissue interface insert (FIG. 3C), according to embodiments of the present disclosure.

FIG. 4 is a graph illustrating the growth curves of various strains of bacteria, according to an embodiment of the present disclosure.

FIG. 5 is a graph illustrating the effect of gNO pressure on bioburden in treated and untreated tissue, according to an embodiment of the present disclosure.

FIG. 6 is a graph illustrating the effect of gNO exposure time on bioburden in treated and untreated tissue, according to an embodiment of the present disclosure.

FIG. 7 is a graph illustrating a minimum amount of gNO exposure time to achieve maximum bioburden reduction, according to an embodiment of the present disclosure.

FIG. 8 is a graph illustrating the effects of reduced gNO pressure on bioburden in treated and untreated tissue, according to an embodiment of the present disclosure.

FIG. 9 is a graph illustrating the effects of gNO pressure, exposure time, and flow on tissue viability, according to an embodiment of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure and/or the claims.

### DETAILED DESCRIPTION

Embodiments of the present disclosure provide materials and methods relating to cell and tissue culture testing systems. Certain embodiments of the present disclosure relate to in vitro testing systems that are useful for performing experiments to investigate the potential for various factors to reduce bioburden, reduce the manifestations of infection, and to promote wound healing in cultured cells and tissues.

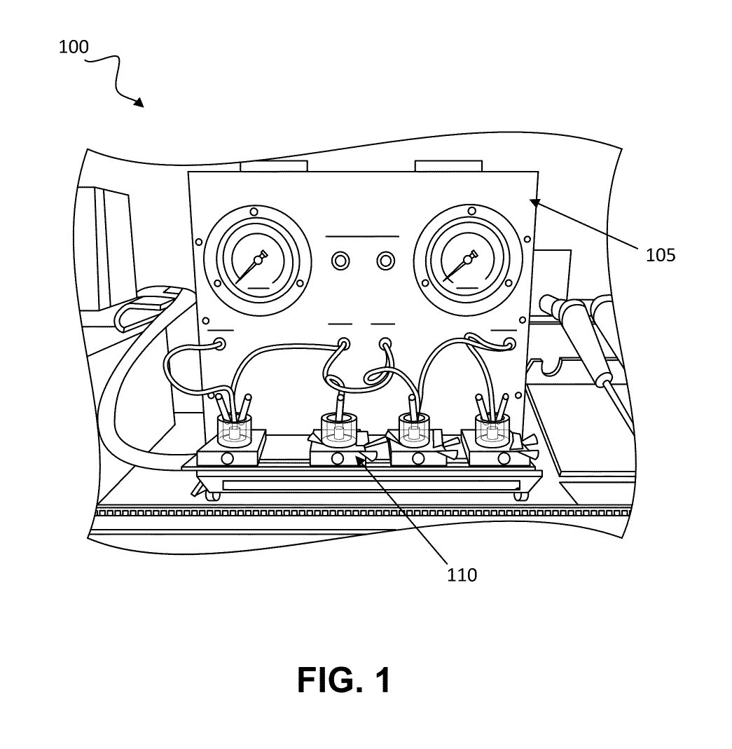

FIG. 1 is a representation of a tissue culture testing system 100 that includes a gaseous Nitric Oxide (gNO) delivery device 105 functionally coupled to one or more tissue culture testing apparatuses 110. In some embodiments, the gNO delivery device is a gas manifold device and the tissue culture testing apparatus includes at least one Franz cell chamber (or Franz cell). In some embodiments, the Franz cell chamber can be modified such that gNO flows from the gNO delivery device to the cells and tissues within the Franz cell at various pressures, flow rates, and concentrations during an experimental process. The gNO delivery device can also be equipped with a gas flow regulator that measures flow rate of the gNO and a gas pressure regulator that measures pressure of the gNO as the gNO flows through the tissue culture testing system 100. Franz cells can be coupled to the gNO delivery device in series, in parallel, or in combinations of series and parallel, depending on the experimental parameters and protocol being used. Cells and tissues contained within individual Franz cells can be arranged so that they are treated as replicates in a given experimental protocol, or they can be arranged so that separate experimental protocols can be conducted within each Franz cell.

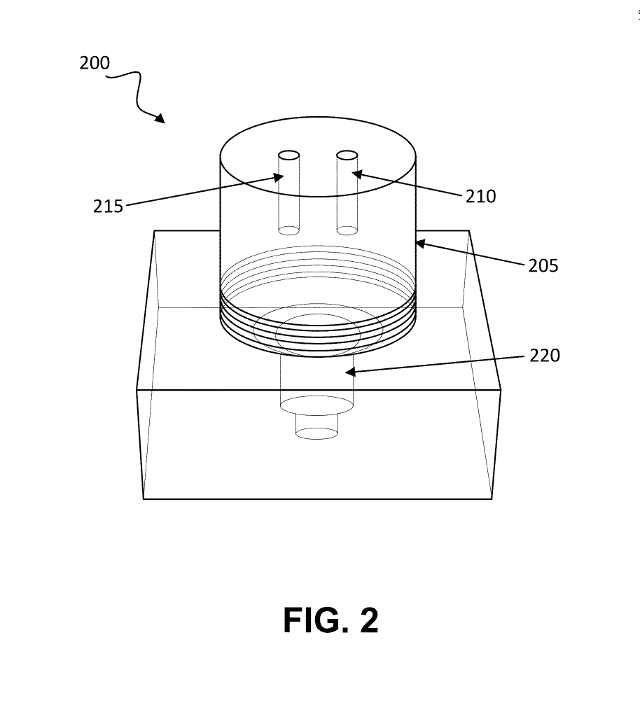

As shown in FIG. 2, individual Franz cells 200 generally include two primary chambers separated by a membrane. The top chamber 205 can include various structures that allow for the input and/or alteration of experimental conditions. For example, the top chamber 205 can include one or more gas inlets 210 and one or more gas outlets 215 for the application of pressurized gasses, such as gNO, to cells or tissue samples. The bottom chamber 220 of Franz cell 200 is generally used to contain and supply liquid media and nutrients to cells or tissue samples. In some aspects, the bottom chamber 220 can be configured to have various input structures to allow for the replacement and/or sampling of media and/or cells and tissues at regular intervals while carrying out an experimental protocol. In some aspects, Franz cell 200 can be configured to allow for the sampling and recording of changes in certain experimental factors, including, for example, pH and nitrite levels, while an experimental protocol is being conducted. In some aspects, the cells or tissue samples can be removed from a Franz cell and subjected to further experimental procedures, such as tissue homogenization procedures.

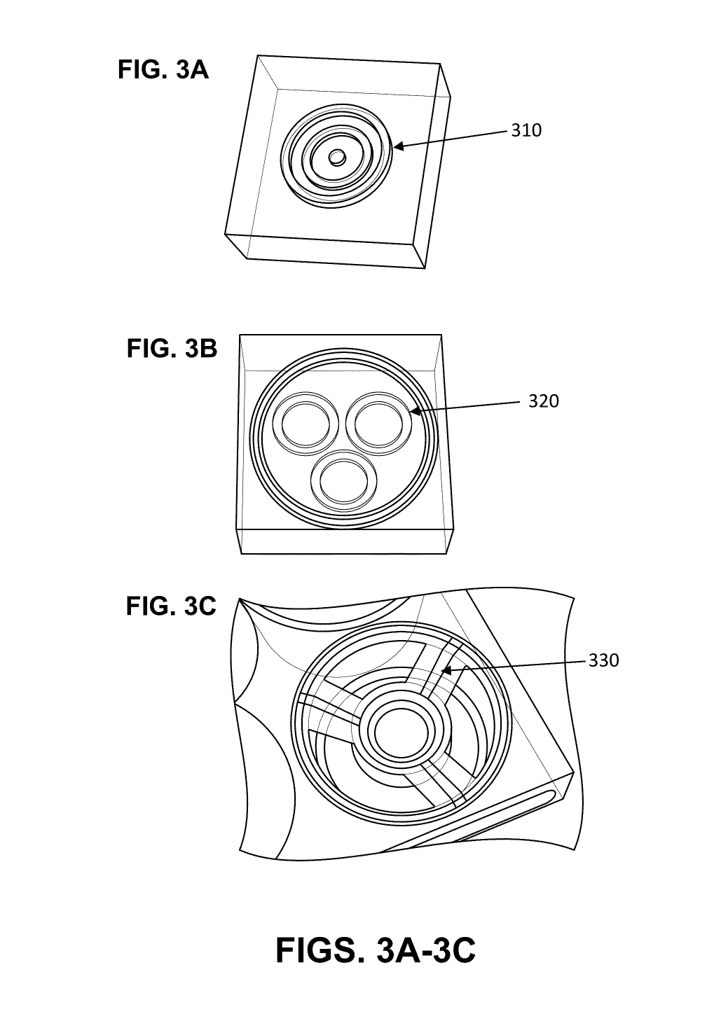

As shown in FIG. 3, the bottom chamber of a Franz cell can be a single chamber 310 configured to house media for a single sample of cells or tissues (FIG. 3A), or the bottom chamber of a Franz cell can be divided into two or more subchambers 320 configured to house media for different samples and/or treatments (FIG. 3B). In some aspects, as shown in FIG. 3B, the use of a Franz cell that includes three sample subchambers allows for experiments to be conducted in triplicate under nearly identical experimental conditions (e.g., exposed to gNO at the same pressure, flow rate, and exposure time). In this manner of operation, variation in experimental conditions can be reduced, thus increasing the accuracy of the data obtained as well as the efficiency of the experimental process.

In some embodiments, the tissue culture testing systems of the present disclosure can be used to conduct assays involving skin tissue and cells, including infection assays and wound healing assays. The tissue culture testing systems of the present disclosure can be used to investigate the ability of pressurized gNO to reduce bioburden, along with various manifestations of infection caused by a pathogen. In some aspects, the effects of pressure, exposure time, and flow rate on bioburden in a cell or tissue sample may be tested using a NO delivery device coupled to one or more Franz cells (FIG. 1). As used herein, bioburden generally refers to the number of bacteria or other pathogens present on a surface, for example, the surface of a tissue or wound. Reducing bioburden generally correlates with reducing or minimizing an infection, as well as the various symptoms that accompany an infection (e.g., pain, swelling, redness, foul odor, blood or pus being released, etc.). Reducing bioburden and reducing infection also tend to correlate with accelerated wound healing and the growth of healthy tissue. The application of pressurized NO for a given amount of time at a given flow rate can reduce bioburden, which in turn promotes healing. In some aspects, the application of pressurized NO for a given amount of time at a given flow rate can reduce bioburden in a full-thickness skin tissue sample and accelerate or promote the healing of a wound in that skin tissue sample.

The tissue culture testing systems of the present disclosure can also be used to investigate the ability of pressurized gNO to promote wound healing and repair. In some aspects, the membrane separating the top chamber and the bottom chamber of a Franz cell can include a skin tissue sample, such as a full-thickness skin tissue sample used to model various molecular, cellular and biochemical processes taking place within the sample. In some aspects the full-thickness skin tissue model includes normal, human-derived epidermal keratinocytes (NHEK) and normal, human-derived dermal fibroblasts (NHFB) which have been cultured to form a multilayered, highly differentiated model of the human dermis and epidermis.

In some embodiments, the membrane used in the tissue culture testing systems of the present disclosure can be synthetic and/or can include biological tissue, including natural biological tissue that is obtained from a donor (e.g., grafts or explants) and biological tissue that is bioengineered (e.g., engineered tissue equivalents), or combinations of both. Examples of suitable types of natural or bioengineered biological tissue suitable for use in the tissue culture testing systems of the present disclosure include, but are not limited to, skin, lung, tracheal, nasal, placental, vaginal, rectal, colon, gut, stomach, bladder, or corneal tissue. In some aspects, skin tissue, such as hairless mouse skin, porcine skin, guinea pig skin, or human skin can be used. Examples of suitable engineered tissues include, but are not limited to, DERMAGRAFT, a human fibroblast-derived dermal substitute (Smith & Nephew, Inc. of Largo, Fla.), and EPIDERM, a skin model from human-derived epidermal keratinocytes available from MatTek Corporation (Ashland, Mass.). Examples of synthetic membranes include, but are not limited to, elastomeric membranes, polymeric membranes, polyethersulfone (PES) membranes, low-density polyethylene (LDPE) membranes, cellulose acetate membranes, silicone membranes, hydrophobic polyvinylidene fluoride (PVDF) membranes, polycarbonate membranes, chitosan membranes, composite cellophane membranes, poly (dimethylsiloxane) membranes, cellulose nitrate membranes and the like.

In some aspects, as shown in FIG. 3C, a Franz cell can include a tissue interface insert 330 that holds the cells or tissue on which experiments will be conducted. The tissue interface insert 330 can engage the Franz cell at the bottom portion of the bottom chamber (e.g., snapped into the bottom portion of the bottom chamber), and a seal can be established to prevent the outflow of media from the bottom chamber. In some aspects, a Franz cell can include a tissue interface insert 330 having two or more membranes corresponding to two or more subchambers in the bottom chamber of the Franz cell (see FIG. 3B). In some aspects, a Franz cell can include a tissue interface insert 330 having three membranes corresponding to three subchambers in the bottom chamber of the Franz cell. In this configuration, a particular set of experimental parameters can be tested in triplicate within a single Franz cell, thus reducing experimental variation. For example, this configuration can be used to test the ability of gNO delivered at various pressures, flow rates, and concentrations to reduce bioburden in a full-thickness skin tissue sample. Other configurations and arrangements of Franz cells can also be used with the various embodiments of the tissue culture testing systems of the present disclosure, as would be recognized by one of ordinary skill in the art based on the present disclosure.

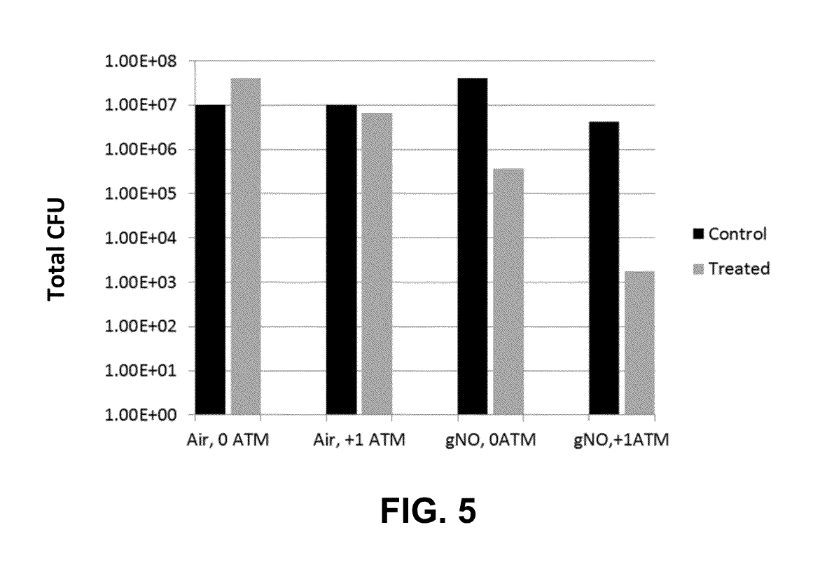

Integrating Franz cells into an NO delivery device allows for the testing of various experimental factors or perturbations on cells and tissue samples. For example, a NO delivery device that includes one or more Franz cells allows for the testing of various levels of pressurized gNO up to 1 atmosphere (ATM), or 14.695 pounds-force per square inch (psi), independent of, and in addition to, the pressure of the external environment (e.g., barometric pressure). Such systems also allow for the testing of NO flow rates and exposure times on cells and tissue samples. The systems of the present disclosure are also generally configured to deliver air, or another suitable gas that can be used as a control, for the purposes of conducting properly controlled experiments having independent and dependent variables and for performing accurate comparisons among treatment groups (see FIG. 5). Franz cells can be equipped to allow for constant readings to be obtained during experimentation, or Franz cells can be equipped to obtain readings only at the end of an experimental protocol. For example, an NO delivery system coupled to one or more Franz cells can be used to sample pH and nitrate levels of homogenized full-thickness tissue at the end of an experimental protocol involving exposing the tissue to pressurized NO for a given amount of time, at a given concentration, and at a given flow rate.

For example, a NO delivery device coupled to a plurality of Franz cells can be used to investigate the effects of exposing a full-thickness skin model to pressurized NO for a given period of time and at a given flow rate in order to determine how these experimental factors reduce bioburden in the sample. Any pathogen can be used to infect the tissue samples, including but not limited to, bacteria, fungi, viruses, protozoans and the like. In some aspects, the pathogen is a bacterial pathogen, including but not limited to, Staphylococcus, MRSA, Acinetobacter and Pseudomonas. In some aspects, tissues can be infected with a bacteria or other pathogen for about 3 hours to model an acute infection scenario. In other aspects, tissues can be infected with a bacterial or other pathogen for about 24 hours to model a chronic infection scenario. In other aspects, the NO delivery device coupled to a plurality of Franz cells can be used to model wound repair and bioburden reduction in the context of various other types of wounds and disease indications, including but not limited to, burns, wrinkles, surgical wounds, trauma wounds, abscesses, actinic keratosis, keloids, scars, skin cancer and the like.

After an infection period is over, tissue samples can be homogenized, and serial dilutions can be grown on LB agar plates in order to facilitate the counting of colony formation units (CFUs), which approximate the level of bacteria present in the sample after infection. Other methods of quantifying infection and the various manifestations of infection are also possible, as would be recognized by one of ordinary skill in the art based on the present disclosure. For example, activation of various cytokines can be measured, as well as gene expression (upregulation and/or down regulation) of various genetic regulators of inflammatory and immune responses taking place in cells and tissues. These experimental procedures allow for the investigation of various experimental factors and perturbations, such as NO pressures, exposure times, and flow rates, in the context of skin infection and how they may influence bioburden reduction and wound healing.

The various experimental parameters being investigated using the tissue culture systems of the present disclosure may vary, depending on the bacteria or pathogen being investigated. For example, tissue samples can be exposed to gasses, chemicals, liquids solutions, hormones, cell signaling molecules, environmental contaminants and the like, depending on the nature of the experiments being conducted. Tissue samples can be exposed to various experimental perturbations and/or substances at various concentrations and pressures. Tissue samples can be exposed to substances (e.g., gases) at concentrations that range from, for example, about 1 part per million (ppm) or about 0.0001% to about 100,000 ppm or about 10%. In some embodiments, the gNO delivered to cells and tissues is part of a gas mixture that has a concentration of NO that ranges from about 1 ppm to about 1500 ppm, from about 1000 ppm to about 5000 ppm, from about 4000 ppm to about 10,000 ppm, from about 9,000 ppm to about 16,000 ppm, from about 15,000 ppm to about 22,000 ppm, from about 21,000 ppm to about 28,000 ppm, from about 27,000 ppm to about 34,000 ppm, and from about 33,000 ppm to about 40,000 ppm. In some aspects, the gNO delivered to the subject is 10,000 ppm, or about 1.0% of the gas mixture (1 ppm is about 0.0001%). Tissue samples can be exposed to various substances and/or perturbations for varying amounts of time, including seconds, minutes, hours, and days. Tissue samples can be exposed to various gasses at various pressures ranging from about 0 ATM to about 1 ATM (independent of and in addition to the pressure applied by the external environment). Additionally, the tissue culture systems of the present disclosure can be used with any tissue sample or cell sample, including, but not limited to the various cells and tissues that are included in or associate with the integumentary system.

In some embodiments, the NO testing devices of the present disclosure can be used to deliver gNO at pressures anywhere between about 0 atmospheres (ATM) to about 1 ATM (i.e., the pressure within the subject interface unit). The delivery of gNO to a subject in this range is independent of, and in addition to, the pressure of the external environment (e.g., barometric pressure). As would be recognized by one of ordinary skill in the art based on the present disclosure, units of pressure can be expressed using various metrics, including ATMs, pounds-force per square inch (e.g., lbf/in² or psi), bar (e.g., Mbar, kilobar, millibar, etc.), pascal (e.g., Pa, kPa, MPa, etc.) and/or ton (e.g., Torr, mTorr, etc.). For example, 1 ATM can be expressed as 14.695 psi. In some aspects of the present disclosure, pressure can be measured and expressed in increments that are tenths, hundredths and/or thousandths of these various metrics. In some aspects, the gNO is delivered at various ranges. For example, the gNO gas can be delivered at pressures from about 0 ATM to about 1.0 ATM, from about 0 ATM to about 0.9 ATM, from about 0 ATM to about 0.8 ATM, from about 0 ATM to about 0.7 ATM, from about 0 ATM to about 0.6 ATM, from about 0 ATM to about 0.5 ATM, from about 0 ATM to about 0.4 ATM, from about 0 ATM to about 0.3 ATM, from about 0 ATM to about 0.2 ATM, and from about 0 ATM to about 0.1 ATM. In some aspects, the gNO can be delivered at pressures from about 0.1 ATM to about 0.5 ATM, from about 0.15 ATM to about 1.0 ATM, from about 0.15 ATM to about 0.5 ATM, from about 0.15 ATM to about 0.25 ATM, and from about 0.25 ATM to about 0.5 ATM. In some aspects, the gNO can be delivered at pressures of about 0.1 ATM, about 0.15 ATM, about 0.2 ATM, about 0.25 ATM, about 0.3 ATM, about 0.35 ATM, about 0.4 ATM, about 0.45 ATM, about 0.5 ATM, about 0.55 ATM, about 0.6 ATM, about 0.65 ATM, about 0.7 ATM, about 0.75 ATM, about 0.8 ATM, about 0.85 ATM, about 0.9 ATM, and about 0.95 ATM. In some aspects, administering NO at a pressure ranging from about 0.1 ATM (1.47 psi) to about 0.35 ATM (5.144 psi) is sufficient to reduce bioburden in skin tissue or cells, thereby reducing the manifestations of infection. In some aspects, administering NO at a pressure ranging from about 0.1 ATM (1.47 psi) to about 0.3 ATM (4.409 psi) is sufficient to reduce bioburden in skin tissue or cells, thereby reducing the manifestations of infection. In some aspects, administering NO at a pressure ranging from about 0.1 ATM (1.47 psi) to about 0.25 ATM (3.674 psi) is sufficient to reduce bioburden in skin tissue or cells, thereby reducing the manifestations of infection. Administering NO in these pressure ranges is sufficient to reduce bioburden without significantly compromising the viability of the healthy skin cells.

In some embodiments, the NO testing systems of the present disclosure can be used to administer NO to cells and tissues at a certain flow rate. As would be recognized by one of ordinary skill in the art based on the present disclosure, units of flow rate can be expressed using various metrics, including liters/minute (LMP) and/or cubic centimeters per minute (cm³/min or cc/min). For example, NO can be delivered at a flow rate ranging from about 0.1 liters/minute to about 2.0 liters/minute, from about 0.1 liters/minute to about 1.9 liters/minute, from about 0.1 liters/minute to about 1.8 liters/minute, from about 0.1 liters/minute to about 1.7 liters/minute, from about 0.1 liters/minute to about 1.6 liters/minute, from about 0.1 liters/minute to about 1.5 liters/minute, from about 0.1 liters/minute to about 1.4 liters/minute, from about 0.1 liters/minute to about 1.3 liters/minute, from about 0.1 liters/minute to about 1.2 liters/minute, from about 0.1 to about 1.1 liters/minute, from about 0.1 liters/minute to about 1.0 liters/minute, from about 0.1 liters/minute to about 0.9 liters/minute, from about 0.1 liters/minute to about 0.8 liters/minute, from about 0.1 liters/minute to about 0.7 liters/minute, from about 0.1 liters/minute to about 0.6 liters/minute, from about 0.1 liters/minute to about 0.5 liters/minute, from about 0.1 liters/minute to about 0.4 liters/minute, from about 0.1 liters/minute to about 0.3 liters/minute, and from about 0.1 liters/minute to about 0.2 liters/minute. In some aspects, the NO can be delivered at a flow rate of about 0.1 liters/minute, about 0.2 liters/minute, about 0.3 liters/minute, about 0.4 liters/minute, about 0.5 liters/minute, about 0.6 liters/minute, about 0.7 liters/minute, about 0.8 liters/minute, and about 0.9 liters/minute, about 1.0 liters/minute, about 1.2 liters/minute, about 1.3 liters/minute, about 1.4 liters/minute, about 1.5 liters/minute, about 1.6 liters/minute, about 1.7 liters/minute, about 1.8 liters/minute, about 1.9 liters/minute, and about 2.0 liters/minute.

In some embodiments, the NO testing systems of the present disclosure can be used to administer NO to cells and tissues for a certain period of time. For example, NO can be delivered for a period of time ranging from about 30 minutes to about 180 minutes, from about 30 minutes to about 170 minutes, from about 30 minutes to about 160 minutes, from about 30 minutes to about 150 minutes, from about 30 minutes to about 140 minutes, from about 30 minutes to about 130 minutes, from about 30 minutes to about 120 minutes, from about 30 minutes to about 110 minutes, from about 30 minutes to about 90 minutes, from about 30 minutes to about 80 minutes, from about 30 minutes to about 70 minutes, from about 30 minutes to about 60 minutes, from about 30 minutes to about 50 minutes, and from about 30 minutes to about 40 minutes. In some aspects, NO can be delivered for a period of time of about 110 minutes, about 105 minutes, about 100 minutes, about 95 minutes, about 90 minutes, about 85 minutes, about 80 minutes, about 75 minutes, about 70 minutes, about 65 minutes, about 60 minutes, about 55 minutes, about 50 minutes, about 45 minutes, about 40 minutes, about 35 minutes, about 30 minutes, about 25 minutes, about 20 minutes, about 15 minutes, about 10 minutes, and about 5 minutes.

In some embodiments, the NO testing devices and systems of the present disclosure can include one or more gas sensors (e.g., electrochemical sensors) for measuring the concentration of one or more gases being delivered to cells and tissues. For example, the testing systems of the present disclosure can include nitric oxide sensors, nitric dioxide sensors, and/or oxygen sensors. These sensors can be functionally coupled to the source of the gas (e.g., NO tank or cylinder) and/or they can be coupled to one or more of the Franz cells to measure gas concentrations at the site of the cells and tissues being tested. In some aspects, gas sensors can help to maintain a constant flow rate and concentration of NO over a given experimental period.

Embodiments of the present disclosure also include methods for performing experiments using the tissue culture testing systems of the present disclosure on cells and tissue samples in order to investigate the effects of pressurized gNO on reducing bioburden, reducing the manifestations of an infection, and/or promoting wound healing. In some aspects, the method includes placing a cell or tissue sample on a membrane of a tissue culture testing apparatus, such as a Franz cell. The tissue culture testing apparatus can be structured such that the membrane separates a top chamber from a bottom chamber in the tissue culture testing apparatus. The method also includes infecting a tissue sample with one or more pathogens, such as a bacterial pathogen, and delivering pressurized gaseous gNO to the infected tissue sample using a gNO delivery device according to a predetermined experimental protocol. The gNO delivery device is typically functionally coupled to the tissue culture testing apparatus. The method also includes determining the extent of bioburden reduction in the infected tissue sample, as compared to a control sample. As described above, the tissue sample used can be is a full-thickness skin tissue sample that is coupled to the membrane, and the method can include delivering gNO according to a predetermined experimental protocol. The experimental protocol depends on the specific variables being tested, which may include, but not limited to, the concentration, flow rate, and pressure at which the gNO is being delivered to the tissue sample. In some aspects, determining the extent of bioburden reduction in a treated tissue sample involves comparing the treated tissue sample to an untreated control tissue sample prior to, and upon completion of, the predetermined experimental protocol. According to the method, determining the extent of bioburden reduction in the treated tissue sample can include comparing total CFUs from the treated tissue sample to total CFUs from the untreated control tissue sample prior to, and upon completion of, the predetermined experimental protocol.

### EXAMPLES

Examples of the present disclosure are included to demonstrate certain embodiments presented herein. It should be appreciated by those of skill in the art that the techniques disclosed in the examples that follow represent techniques discovered to function well in the practices disclosed herein. However, those of skill in the art should, in light of the present disclosure, appreciate that many changes can be made in the certain embodiments which are disclosed and still obtain a like or similar result without departing from the spirit and scope herein.

### Bacteria Growth Curves

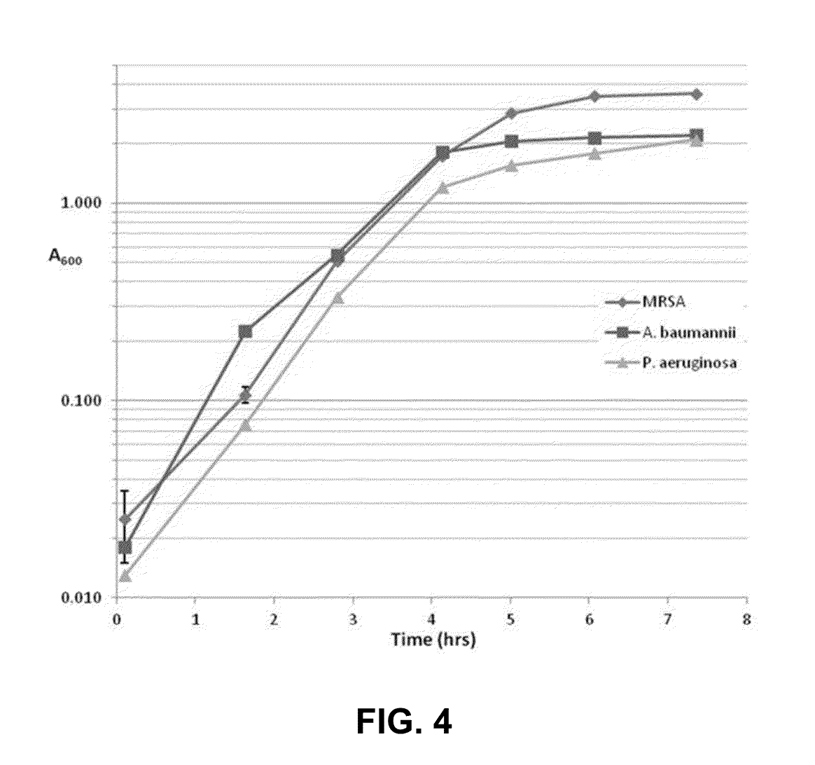

In FIG. 4, the growth curves of several pathogenic bacteria are shown, including MRSA, Acinetobacter and Pseudomonas. In order to determine whether a given set of experimental factors or perturbations relating to NO exposure reduces bioburden, tissue samples were infected with the same concentration of bacteria under the same experimental conditions. In this case, bacteria were be grown to an optical density (OD) of about 1×10⁷ CFUs, as shown in FIG. 4, and about 10-50 μl of that bacteria culture was used to infect a given cell or tissue sample. In some aspects, 15 μl of a bacterial culture is sufficient to infect a cell or tissue sample.

### The Effects of gNO at Various Pressures on Bioburden Reduction

As shown in FIG. 5, the effects of gNO exposure time on bioburden in treated (infected and exposed to gNO) and untreated (infected and exposed to air) tissue samples were tested in a NO delivery device coupled to a plurality of Franz cells. Full-thickness skin tissue samples (e.g., EPIDERM tissue model from MatTek Corp., Ashland, Mass. 01721) were exposed to various experimental factors. As shown, exposure of the skin tissue samples to pressurized air at 0 ATM or 1 ATM (independent of and in addition to the pressure applied by the external environment) for 90 minutes at a flow rate of 0.1 liters/minute did not cause a significant reduction in bioburden. (Total CFUs are represented along the y-axis, while gas pressure is represented on the x-axis.) However, exposing full thickness skin tissue samples to pressurized gNO at 1 ATM (as compared to 0 ATM) did significantly reduce bioburden. The skin samples used were infected with S. aureus for 24 hours, followed by 90 minutes of pressurized exposure at a flow rate of 0.1 liters/minute.

### Eradication of Various Pathogens Using Pressurized gNO

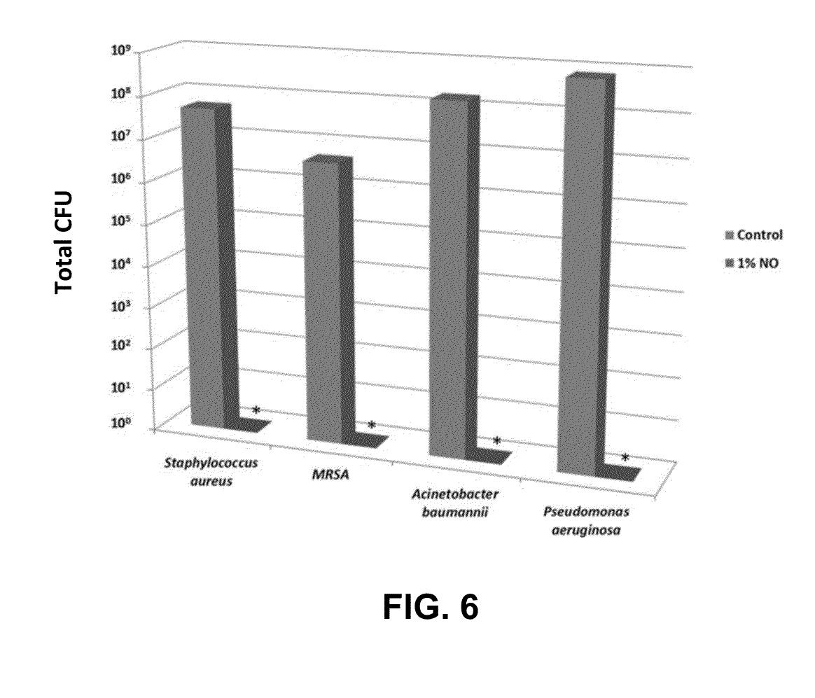

As shown in FIG. 6, the effects of pressurized gNO on bioburden in treated and untreated tissue samples were tested in a NO delivery device coupled to a plurality of Franz cells. Exposing full-thickness skin tissue samples to 1% pressurized NO at 1 ATM (independent of and in addition to the pressure applied by the external environment) for 120 minutes at a flow rate of 0.1 liters/minute completely eliminated (i.e., no CFUs detected) bacterial bioburden after 24 hours of infection (right bar in each pair). Exposing the tissue samples to air at 1 ATM (independent of and in addition to the pressure applied by the external environment) for 120 minutes at a flow rate of 0.1 liters/minute (controls) did not cause a significant reduction in bioburden (left bar in each pair). The samples used were infected with Staphylococcus, MRSA, Acinetobacter or Pseudomonas. These data indicate that pressurized gNO can effectively eradicate a variety of pathogenic bacteria known to cause injection and prevent wound healing, and suggest that longer exposure time can enhance the ability of pressurized NO to reduce bioburden.

### Determining Minimum Exposure Time

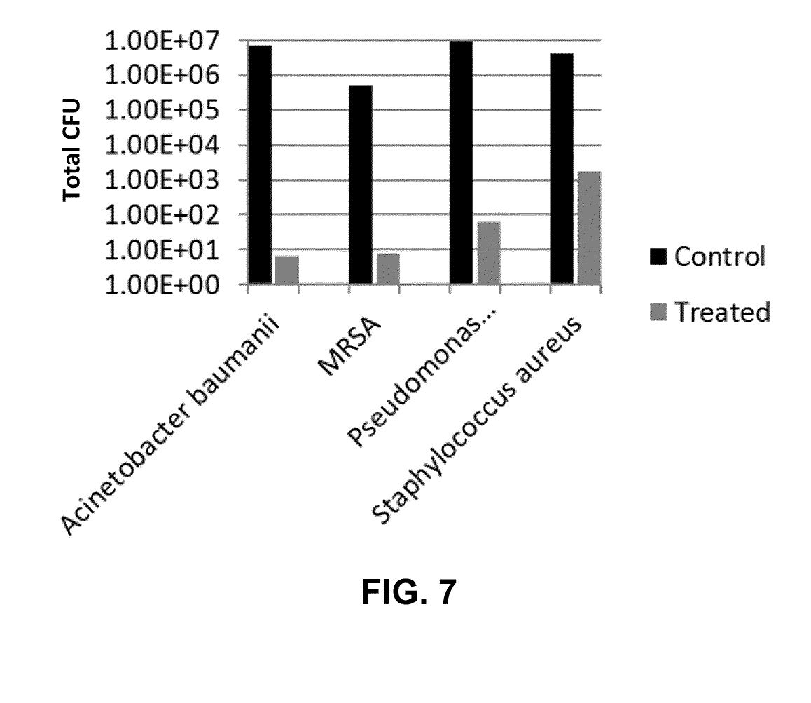

Experiments were also conducted as described above in the previous examples to determine the minimum amount of exposure time necessary to obtain maximum reductions in bioburden. In some cases, determining the minimum tissue exposure time to gNO can reduce any potentially detrimental effects on the health of the tissue, while still reducing bioburden. Flow rate was maintained at 0.1 liters/minute, but the exposure time was shortened to 60 minutes at 1 ATM (independent of and in addition to the pressure applied by the external environment). These parameters were effective against gram negative organisms (A. baumannii and P. aeruginosa); however, gram positive strains (MRSA and S. aureus) did not show significant reduction of bioburden when compared to controls (not shown). At 90 min of exposure, keeping pressure at 1 ATM and flow at 0.1 liters/minute, there was approximately a 5-6 log reduction in A. baumannii, P. aeruginosa and MRSA, while the biofilm-forming S. aureus showed approximately a 3 log reduction (FIG. 7). These data indicate that skin samples infected for 24 hours and subsequently exposed to 1% NO at 1 ATM for 90 minutes at a flow rate of 0.1 liters/minute had significantly reduced bioburden levels in each of the four types of bacteria tested.

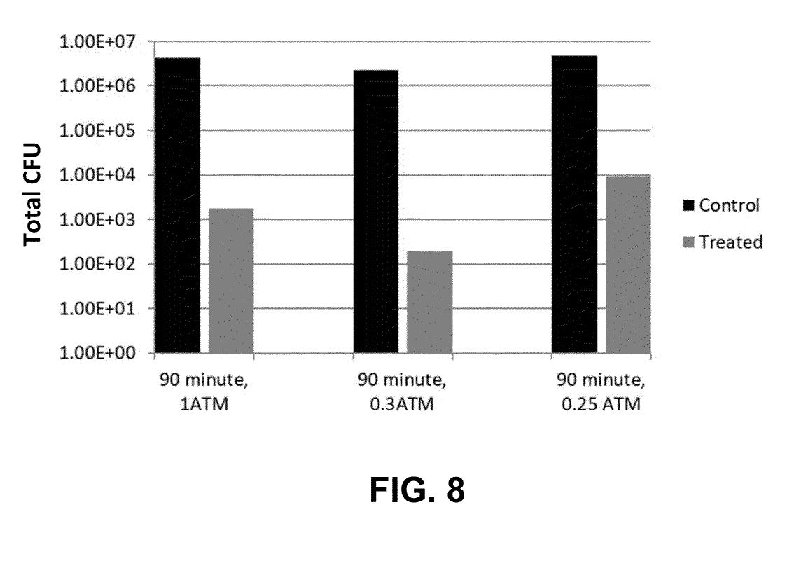

Experiments were also conducted as described above in the previous examples to determine the minimum amount of gNO pressure necessary to obtain maximum reductions in bioburden. In some cases, determining the minimum amount of gNO pressure can reduce any potentially detrimental effects on the health of the tissue, while still reducing bioburden. As shown in FIG. 8, without changing exposure time (90 minutes) or flow rate (0.1 liters/minute), bioburden was significantly reduced in skin samples infected for 24 hours with A. aureus at NO pressures lower than the 1 ATM used in other experiments. NO exposure at 0.3 ATM and 0.25 ATM were sufficient to reduce bioburden in these samples. These data indicate that pressurized gNO can effectively reduce bioburden, even at pressures below 1 ATM (independent of and in addition to the pressure applied by the external environment), which could have beneficial ramifications for the application of the methods and systems of the present disclosure to an in vivo context.

Experiments were also conducted to investigate the effects of various NO pressures, exposure times, and flow rates on tissue viability using MTT assays. An MTT assay is a colorimetric assay for assessing cell viability. NAD(P)H-dependent cellular oxidoreductase enzymes may, under defined conditions, reflect the number of viable cells present. These enzymes are capable of reducing the tetrazolium dye MTT 3-(4,5-dimethylthiazol-2-yl)-2,5-diphenyltetrazolium bromide to its insoluble formazan, which has a purple color. Other closely related tetrazolium dyes including XTT, MTS and the WSTs, can also be used. Tetrazolium-based dye assays can also be used to measure cytotoxicity (loss of viable cells) or cytostatic activity (shift from proliferation to quiescence) of potential medicinal agents and toxic materials.

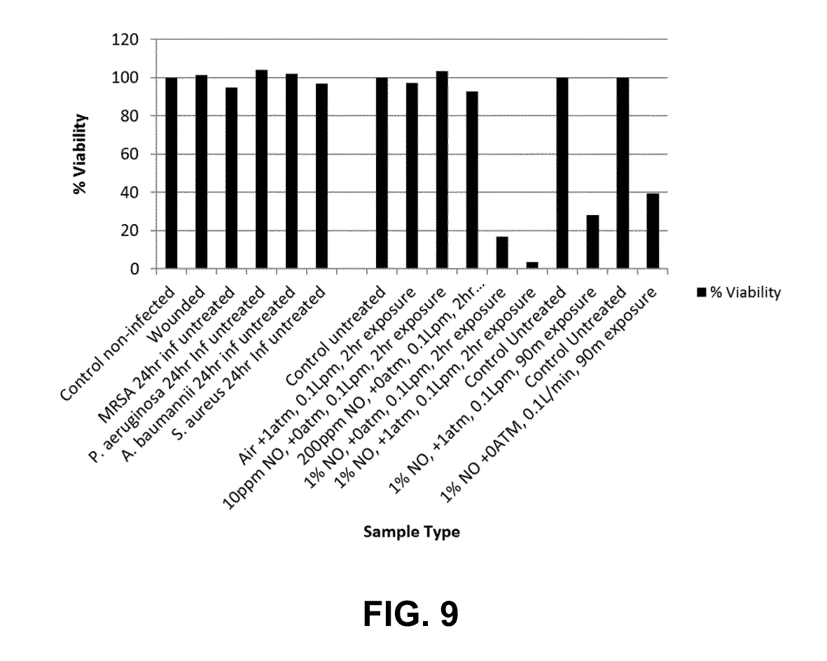

As shown in FIG. 9, tissue viability assessed using MTT assays was not significantly affected by the infection itself (see, e.g., columns 3-6) when compared to wounded only and non-infected tissues (see, e.g., columns 1 and 2). Pressure itself also does not significantly affect viability (see, e.g., columns 7 and 8). However, the combination of pressurized gNO and increasing concentrations of NO caused a decrease in viability (e.g., columns 8-13). Additionally, as exposure time decreased, viability increased in the presence of 1% NO with or without pressure (see, e.g., columns 13-16).

There are several factors that affect tissue viability in an in vitro testing system that may not be present in an in vivo setting. For example, tissue in in vitro culture systems generally lack the ability to clear detrimental bi-products involved in NO physiology, including nitrites and nitrates, whereas these bi-products are easily eliminated in vivo. Additionally, the pH levels in in vitro systems are not as tightly regulated as in vivo. To better appreciate these differences, experiments were conducted in which pH and nitrite levels were measured from tissue homogenates after infection with various strains of bacteria for either 3 or 24 hours. The results are represented in Table 1 below.

TABLE 1
pH and nitrate levels after NO treatment.

| | | | Nitrites (μmol) |
|---|---|---|---|
| | | pH | after treatment |
| | | after treatment | 90 min, 1 |
| | | 90 min, 1 ATM, 1% | ATM, 1% NO |
| | Infection | NO or no treatment | or no treatment |
| Pathogen | time | (Control) | (Control) |
| Staphylococcus aureus | 3 hrs | 3.54 | 7.55 |
| Control S. aureus | 3 hrs | 7.48 | 0 |
| Staphylococcus aureus | 24 hrs | 4.00 | 8.22 |
| Control S. aureus | 24 hrs | 7.49 | 0 |
| MRSA | 3 hrs | 3.79 | 6.44 |
| Control MRSA | 3 hrs | 7.46 | 0 |
| MRSA | 24 hrs | 3.87 | 9.66 |
| Control MRSA | 24 hrs | 6.95 | 0 |
| A. baumannii | 3 hrs | 3.93 | 8.77 |
| Control A. baumannii | 3 hrs | 7.67 | 0 |
| A. baumannii | 24 hrs | 3.93 | 15.88 |
| Control A. baumannii | 24 hrs | 7.72 | 0 |
| P. aeruginosa | 3 hrs | 3.98 | 8.44 |
| Control P. aeruginosa | 3 hrs | 7.64 | 0 |
| P. aeruginosa | 24 hrs | 4.11 | 17.00 |
| Control P. aeruginosa | 24 hrs | 7.51 | 0 |

The present disclosure, in various aspects, embodiments, and configurations, includes components, methods, processes, systems and/or apparatus substantially as depicted and described herein, including various aspects, embodiments, configurations, sub combinations, and subsets thereof. Those of skill in the art will understand how to make and use the various aspects, aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, e.g., for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A tissue culture testing system comprising:
a gaseous nitric oxide (gNO) delivery device;
a tissue culture testing apparatus, the tissue culture testing apparatus comprising at least one top chamber and at least one bottom chamber; and
a membrane separating the at least one top chamber from the at least one bottom chamber;
wherein the gNO delivery device is functionally coupled to the tissue culture testing apparatus and delivers pressurized gNO to a tissue sample.

2. The system of claim 1, further comprising at least one gas flow regulator and at least one gas pressure regulator.

3. The system of claim 1, further comprising a source of gNO functionally coupled to the gNO delivery device.

4. The system of claim 1, wherein the gNO delivery device further comprises one or more nitric oxide sensors.

5. The system of claim 1, wherein the gNO delivery device further comprises one or more oxygen sensors.

6. The system of claim 1, wherein the top chamber of the tissue culture testing apparatus comprises at least one gas inlet and at least one gas outlet.

7. The system of claim 1, wherein the bottom chamber of the tissue culture testing system comprises two or more subchambers.

8. The system of claim of claim 1, wherein the membrane separating the top chamber and the bottom chamber of the tissue culture testing apparatus is coupled to a tissue interface insert that engages the bottom chamber of the tissue culture testing apparatus.

9. The system of claim 1, wherein the membrane comprises the tissue sample, and wherein the tissue sample is a full-thickness skin tissue sample.

10. The system of claim 1, wherein the tissue sample is a full-thickness skin tissue sample, and wherein the full-thickness skin tissue sample is infected with one or more pathogens.

11. The system of claim 1, wherein the tissue sample is a full-thickness skin tissue sample, and wherein the full-thickness skin tissue sample is infected with one or more bacterial pathogens.

12. The system of claim 1, wherein the pressure of the gNO delivered to the tissue sample is from about 0.15 ATM to about 1.0 ATM.

13. The system of claim 1, wherein the gNO is delivered to the tissue sample at a flow rate from about 0.1 liters/minute to about 1.0 liters/minute.

14. The system of claim 1, wherein the concentration of the gNO delivered to the subject is about 1.0%.

15. The system of claim 1, wherein the gNO is delivered to the tissue sample for about 30 minutes to about 120 minutes.

16. The system of claim 1, wherein delivering pressurized gNO to the tissue sample reduces bioburden or reduces one or more manifestations of infection in the tissue sample.

17. A method for determining bioburden reduction in a tissue sample, the method comprising:
placing at least one tissue sample on a membrane of a tissue culture testing apparatus, the membrane separating at least one top chamber from at least one bottom chamber of the tissue culture testing apparatus;
infecting the at least one tissue sample with one or more pathogens;
delivering pressurized gaseous nitric oxide (gNO) to the at least one infected tissue sample using a gNO delivery device according to a predetermined experimental protocol, the gNO delivery device functionally coupled to the tissue culture testing apparatus; and
determining bioburden reduction in the at least one infected tissue sample.

18. The method of claim 17, wherein the tissue sample is a full-thickness skin tissue sample.

19. The method of claim 17, wherein the one or more pathogens is one or more bacterial pathogens.

20. The method of claim 17, wherein the at least one tissue sample comprises at least one treated tissue sample and at least one untreated control tissue sample.

21. The method of claim 17, wherein delivering gNO according to a predetermined experimental protocol comprises delivering gNO at certain concentrations, flow rates, and pressures.

22. The method of claim 17, wherein the at least one tissue sample comprises at least one treated tissue sample and at least one untreated control tissue sample, and wherein the method further comprises delivering gNO to the at least one treated tissue sample and delivering air to the at least one untreated control tissue sample.

23. The method of claim 17, wherein the at least one tissue sample comprises at least one treated tissue sample and at least one untreated control tissue sample, and wherein the method further comprises delivering gNO to both the at least one treated tissue sample and the at least one untreated control tissue sample.

24. The method of claim 23, wherein delivering gNO to both the at least one treated tissue sample and the at least one untreated control tissue sample according to a predetermined experimental protocol comprises delivering gNO at certain concentrations, flow rates, and pressures.

25. The method of claim 17, wherein the at least one tissue sample comprises at least one treated tissue sample and at least one untreated control tissue sample, and wherein determining bioburden reduction in the at least one treated tissue sample comprises comparing the at least one treated tissue sample to the at least one untreated control tissue sample prior to, and upon completion of, the predetermined experimental protocol.

26. The method of claim 17, wherein the at least one tissue sample comprises at least one treated tissue sample and at least one untreated control tissue sample, and wherein determining bioburden reduction in the at least one treated tissue sample comprises comparing total CFUs from the at least one treated tissue sample to total CFUs from the at least one untreated control tissue sample prior to, and upon completion of, the predetermined experimental protocol.
